(19) United States
(12) Patent Application Publication
(10) Pub. No.: US 2022/0168134 A1
(43) Pub. Date: Jun. 2, 2022

(54) Molded Ice Applicator for Biological Surfaces

(71) Applicants: Jasmine Warner, San Marcos, TX (US); Alex Muntefering, San Marcos, TX (US); Chloe Muntefering, San Marcos, TX (US)

(72) Inventors: Jasmine Warner, San Marcos, TX (US); Alex Muntefering, San Marcos, TX (US); Chloe Muntefering, San Marcos, TX (US)

(73) Assignee: Muse Athletics LLC, San Marcos Tx, TX (US)

(21) Appl. No.: 17/110,280

(22) Filed: Dec. 2, 2020

(51) Int. Cl. A61F 7/00; A61H 7/00

(52) U.S. Cl. CPC A61F 7/00; A61H 7/003; A61F 2007/0087; A61H 2201/0107; A61H 2201/0153; A61H 2201/0214; A61H 2201/1253; A61H 2201/1692

(57) ABSTRACT

A cryotherapy device that enables the applicator to massage a biological surface with targeted precision using a sphere of ice attached to a handle and an insulating mold. The device allows for the filling and freezing of water forming a spherical ice form, as well as protection for the applicator's hands and fingers from exposure to the ice. It is comprised of three components: a handle for gripping the device, which is anchored into the spherical ice form, and two sub adjacent hemispherical molds, which seal together in order to contain water that is frozen into a spherical ice form. One hemispherical flexible mold is sub adjacent to the handle, and also serves to provide insulation for the applicator's fingers. The other hemispherical mold possesses rigid protrusions that enable the device to stand vertically on a flat surface. After the water is frozen within the device, this third component is detached from the other two components in order to expose the spherical ice form for application of cryotherapy massage to a biological surface.

### CROSS-REFERENCE TO RELATED APPLICATIONS

Provisional Patent Application No. 62/942,836

### STATEMENT REGARDING FEDERALLY SPONSORED RESEARCH

Not Applicable

### THE NAMES OF THE PARTIES TO A JOINT RESEARCH AGREEMENT

Not Applicable

### STATEMENT REGARDING PRIOR DISCLOSURES BY THE INVENTOR OR A JOINT INVENTOR

Not Applicable

### BACKGROUND OF THE INVENTION

Targeted cryotherapy is a common restorative medical practice. Its use is prevalent among athletes and individuals suffering from pain caused by a variation of factors including impact and muscle fatigue. There are several current methods in the market place including the use of paper and Styrofoam cups which are widely available and cost effective. There are also several products available for ice application that are thermally limiting due to the type of containment material used to keep the cooling substance from escaping the device. Other products are either not ergonomic or contain dangerous amounts of ice which has the potential to lead to overuse and frostbite. Furthermore, the average residential freezer can be limited in space and large cryotherapy devices can pose a problem during normal use.

### FIELD OF THE INVENTION

This field of invention pertains to a thermal massage tool. More specifically a massage tool to apply a molded solidified liquid such as water for the application of cryotherapy or thermal modulation of biological surfaces.

### BRIEF SUMMARY OF THE INVENTION

A massage tool is provided with a mold having a top and bottom, and an application handle coupled to the anchor. The anchor extends from the application handle base to extend into the mold containing the contact surface material in liquid form. The anchor is further engineered with features to increase adhesion to the solidified liquid contact surface material. The anchor can freely move in the contact surface substance while in liquid form until physical change state occurs where the anchor features captivate the solidified contact surface liquid. The massage tool further has the top contact surface mold having a coupling interface with the handle base. A further aspect of this invention is the top mold section comprised of a flexible material allowing for manipulation to fold away from the solidified contact surface revealing additional surface area of the solidified contact surface liquid. While the top mold section is unfolded it also acts as an additional force application surface to work in conjunction with the handle. The handle is coupled with the handle base which is coupled with the anchor. The handle is in cylindrical form to accommodate both right- and left-hand application. Another aspect of this invention is a two-part contact surface liquid solidification mold. This allows for the top section of mold to act as a force manipulation surface as well as an adjustable thermal regulator for the solidified contact surface. The top and bottom sections of the two-part mold have a coupling interface allowing for separation after contact surface liquid has solidified. Further aspects of the two-part mold are orientation stabilizing features on the bottom of the lower mold section. The top section of the mold has an opening to function as both a liquid receiving guided port as well as a handle coupling interface.

### BRIEF DESCRIPTION OF THE SEVERAL VIEWS OF THE DRAWINGS

FIG. 1 Illustrates the side view of the components fully assembled together.

FIG. 2 Illustrates the cross section of the components fully assembled together.

FIG. 3 Illustrates example operations for using an Ice applicator to biological surfaces according to one or more implementations of the disclosed technology.

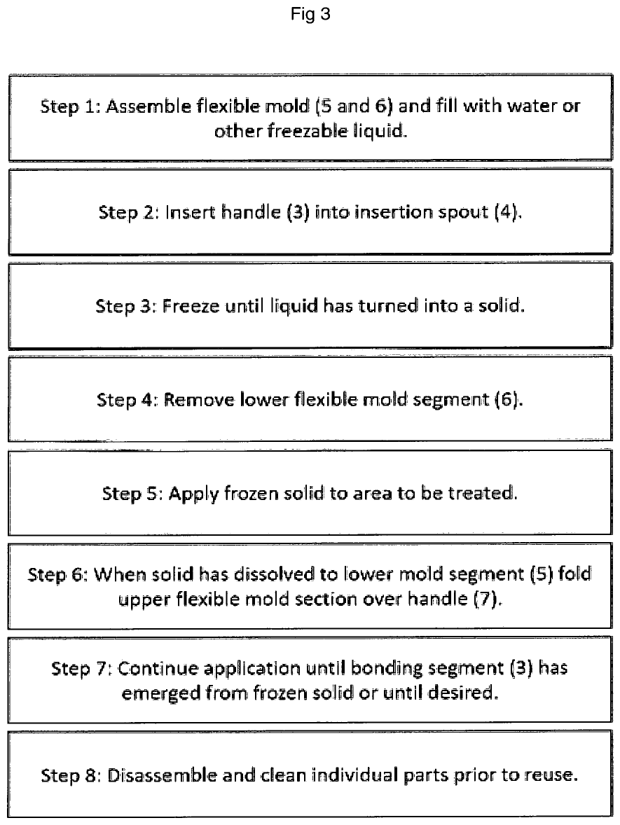

### DETAILED DESCRIPTION OF THE INVENTION

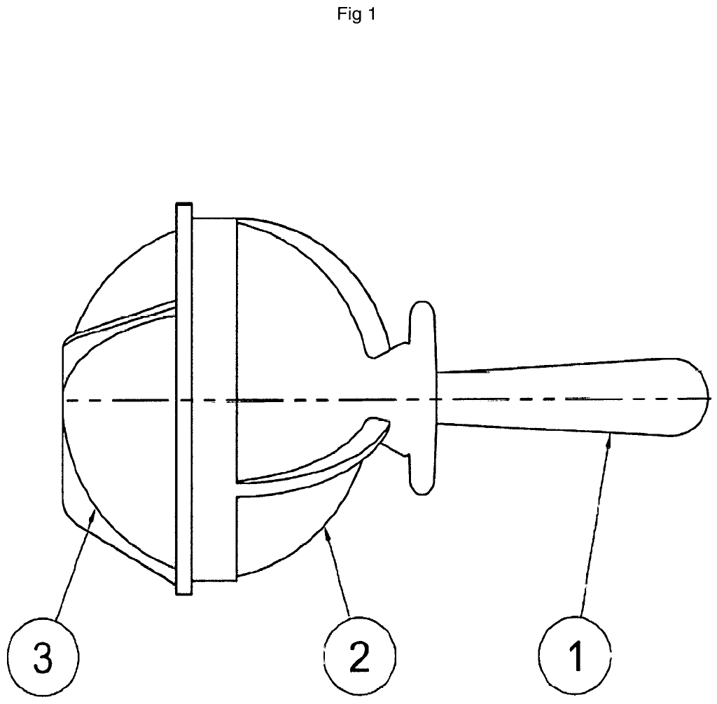

FIG. 1 Illustrates the side view of the components fully assembled together. The assembly includes a 1 handle consisting of a rigid material such as high-density polypropylene or metal, 2 a top mold consisting of flexible material such as silicone and a 3 bottom mold made of either rigid or flexible material.

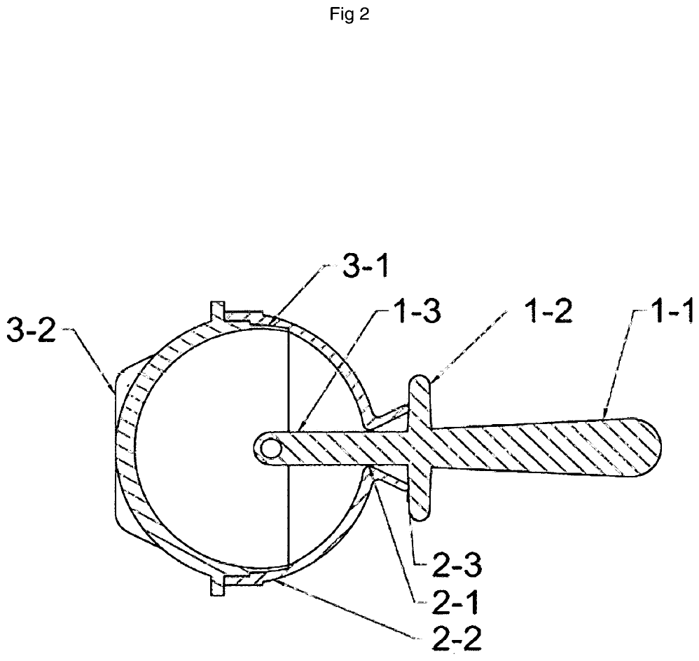

FIG. 2 Illustrates the cross-section view and detailed numbering of its components. The handles section 1-1 interfaces with the applicator's manipulation appendage or modified for a device to assist with adding vibration or force. The 1-2 hilt component helps the applicator provide an additional surface for applying force and acts as an interface to mate to the top mold. The component attached to the hilt opposed to the 1-3 anchor acts as a surface for the enclosed liquid to encapsulate the 1 handle. The next section 2 top mold has a feature 2-3 that acts as a liquid guide during mold filling and as a mating surface for the handle hilt 1-2. The top section of the mold 2-1 can be molded into multiple shapes for different ice applications and can be folded back towards 1-1 to reveal more of the solidified liquid for biological application. The top mold has a mating feature 2-2 that acts like a seal to the bottom mold to contain the liquid until solidified. The 3 bottom mold has a 3-1 feature that mates to the top mold and acts as a seal to contain the liquid until solidified. The bottom mold also has 3-2 features to hold the assembly in the correct orientation with the filling section 2-3 pointing away from the direction of gravity.

1. An Ice applicator consisting of three parts, a rigid handle for device manipulation and application, a flexible top mold that mates with the handle and seals to the bottom mold to act as a cavity for liquid encapsulation.

2. The ice applicator device claim 1 to consist of a flexible top section of mold in sealing position unfolded away from the handle acts as both a top section of the liquid encapsulation cavity, force manipulation surface for increasing biological surface pressure, and a thermal barrier for reduced thermal transfer of the top section of solidified liquid.

3. The ice applicator device claim 1 to consist of a generally hemispheric cavity when the top and bottom molds are mated together but can be designed for alternative sizes and shapes for modulation in application time and force profile.

4. The ice applicator device claim 1 to have features on the opposite side of the sealing edge of the bottom mold to act as feet or structures to mate with a flat surface to hold the fully assembled ice applicator in a position to have the liquid entrance opposite of the gravitational force.

5. A method comprising positioning the handle in the top mating section and corresponding liquid entrance of the top mold section which is sealed to the bottom mold and filling cavity with liquid.

6. The method of claim 5 further comprising of liquid that has solidified by cooling after assembly of components.

7. The method of claim 5, further comprising: gripping the handle above the hilt and anchor sections placing the solidified liquid contact surface to the user or users intended biological surface.

8. The method of claim 5, where the top mold section can be folded back to increase exposed solidified liquid surface area to extend the duration of ice application.

9. The method of claim 5, where the top mold section can be used by the user's hand for increased force of the solidified liquid's surface to the biological surface intended for application.

10. The method of claim 5, where the cavity made by the top and bottom mold have generally a spherical shape but can be modified by using alternatively shaped top and bottom mold sections of different sizes and shapes for alternative application times or surface pressure profile.

11. The method of claim 5, that the top and bottom mold sealing surfaces are the widest part of the cavity.

12. The method of claim 5, that the top mold and bottom mold cavity when sealed has a diameter greater than that of the length of the anchor.

13. The method of claim 5, that the length of the anchor and depth of the mold cavity can be changed to allow for the anchor to signal determined application times for general solidified liquid melt time when in contact with biological surfaces.
